(19) United States
(12) Patent Application Publication
(10) Pub. No.: US 2026/0285854 A1
(43) Pub. Date: Sep. 24, 2026

(54) CRYSTALLINE FORMS OF AN AZETIDINE PARP1 INHIBITOR

(71) Applicant: Gilead Sciences, Inc., Foster City, CA (US)

(72) Inventor: Bing Shi, Redwood City, CA (US)

(21) Appl. No.: 19/387,213

(22) Filed: Nov. 12, 2025

Related U.S. Application Data
(60) Provisional application No. 63/720,535, filed on Nov. 14, 2024.

(51) Int. Cl. C07D 471/04

(52) U.S. Cl. CPC C07D 471/04

(57) ABSTRACT

Described herein are crystalline forms of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound I), or a pharmaceutically acceptable salt or solvate thereof.

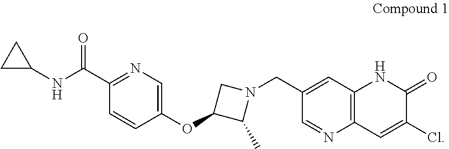 [SMILES: C[C@@H]1[C@@H](Oc2ccc(C(=O)NC3CC3)nc2)CN1Cc1cnc2cc(Cl)c(=O)[nH]c2c1]

### CROSS-REFERENCE TO RELATED APPLICATIONS

This application claims the benefit under 35 U.S.C. 119(e) of U.S. Provisional Application No. 63/720,535, filed on Nov. 14, 2024, which is hereby incorporated by reference in its entirety.

### BACKGROUND

Poly(ADP-ribose)polymerase (PARP) or poly(ADP-ribose)synthase (PARS) has an essential role in facilitating DNA repair, controlling RNA transcription, mediating cell death, and regulating immune response. These actions make PARP inhibitors targets for a broad spectrum of disorders. PARP inhibitors have demonstrated efficacy in numerous models of disease, particularly in models of ischemia reperfusion injury, inflammatory disease, degenerative diseases, protection from adverse effects of cytotoxic compounds, and the potentiation of cytotoxic cancer therapy. PARP has also been indicated in retroviral infection and thus inhibitors may have use in antiretroviral therapy. PARP inhibitors have been efficacious in preventing ischemia reperfusion injury in models of myocardial infarction, stroke, other neural trauma, organ transplantation, as well as reperfusion of the eye, kidney, gut, and skeletal muscle. Inhibitors have been efficacious in inflammatory diseases such as arthritis, gout, inflammatory bowel disease, CNS inflammation such as MS and allergic encephalitis, sepsis, septic shock, hemorrhagic shock, pulmonary fibrosis, and uveitis. PARP inhibitors have also shown benefit in several models of degenerative disease including diabetes (as well as complications) and Parkinson's disease. PARP inhibitors can ameliorate the liver toxicity following acetaminophen overdose, cardiac and kidney toxicities from doxorubicin and platinum based antineoplastic agents, as well as skin damage secondary to sulfur mustards. In various cancer models, PARP inhibitors have been shown to potentiate radiation and chemotherapy by increasing cell death of cancer cells, limiting tumor growth, decreasing metastasis, and prolonging the survival of tumor-bearing animals.

PARP1 and PARP2 are the most extensively studied PARPs for their role in DNA damage repair. PARP1 is activated by DNA damage breaks and functions to catalyze the addition of poly(ADP-ribose) (PAR) chains to target proteins. This post-translational modification, known as PARylation, mediates the recruitment of additional DNA repair factors to DNA lesions.

Following completion of this recruitment role, PARP auto-PARylation triggers the release of bound PARP from DNA to allow access to other DNA repair proteins to complete repair. Thus, the binding of PARP to damaged sites, its catalytic activity, and its eventual release from DNA are all important steps for a cancer cell to respond to DNA damage caused by chemotherapeutic agents and radiation therapy.

Inhibition of PARP family enzymes has been exploited as a strategy to selectively kill cancer cells by inactivating complementary DNA repair pathways. A number of pre-clinical and clinical studies have demonstrated that tumor cells bearing deleterious alterations of BRCA1 or BRCA2, key tumor suppressor proteins involved in double-strand DNA break (DSB) repair by homologous recombination (HR), are selectively sensitive to small molecule inhibitors of the PARP family of DNA repair enzymes. Such tumors have deficient homologous recombination repair (HRR) pathways and are dependent on PARP enzymes function for survival. Although PARP inhibitor therapy has predominantly targeted SRCA-mutated cancers, PARP inhibitors have been tested clinically in non-SRCA-mutant tumors, those which exhibit homologous recombination deficiency (HRD).

It is believed that PARP inhibitors having improved selectivity for PARP1 may possess improved efficacy and reduced toxicity compared to other clinical PARP1/2 inhibitors. It is believed also that selective strong inhibition of PARP1 would lead to trapping of PARP1 on DNA, resulting in DNA double strand breaks (DSBs) through collapse of replication forks in S-phase. It is believed also that PARP1 DNA trapping is an effective mechanism for selectively killing tumor cells having HRD. An unmet medical need therefore exists for effective and safe PARP inhibitors, especially PARP inhibitors having selectivity for PARP1, and a stable form of a PARP1 inhibitor.

### SUMMARY

Disclosed herein are crystalline forms of a L-tartrate salt of Compound I:

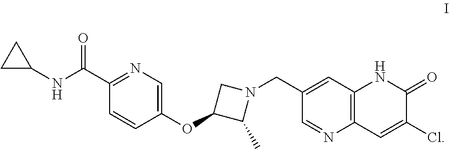 [SMILES: C[C@@H]1[C@@H](Oc2ccc(C(=O)NC3CC3)nc2)CN1Cc1cnc2cc(Cl)c(=O)[nH]c2c1]

Also disclosed herein is a pharmaceutical composition comprising a crystalline form disclosed herein, and a pharmaceutically acceptable excipient.

Also disclosed herein is a method of treating cancer in a subject in need thereof, the method comprising administering a crystalline form disclosed herein.

In some embodiments, the cancer is breast cancer, ovarian cancer, pancreatic cancer, prostate cancer, a hematological cancer, gastrointestinal cancer, or lung cancer.

Also disclosed herein is a method of treating a cancer comprising a BRCA1 and/or a BRCA2 mutation in a subject in need thereof, the method comprising administering a crystalline form disclosed herein.

In some embodiments, the cancer is bladder cancer, brain & CNS cancers, breast cancer, cervical cancer, colorectal cancer, esophagus cancer, Hodgkin lymphoma, non-Hodgkin lymphoma, kidney cancer, leukemia, lung cancer, melanoma, myeloma, oral cavity cancer, ovarian cancer, pancreatic cancer, prostate cancer, skin cancer, stomach cancer, thyroid cancer, or uterus cancer.

### INCORPORATION BY REFERENCE

All publications, patents, and patent applications mentioned in this specification are herein incorporated by reference to the extent applicable and relevant and to the same extent as if each individual publication, patent, or patent application was specifically and individually indicated to be incorporated by reference.

### BRIEF DESCRIPTION OF THE FIGURES

FIG. 1A shows an X-ray powder diffraction (XRPD) pattern for Compound I L-tartrate Form V.

FIG. 1B shows a differential scanning calorimetry (DSC) thermogram for Compound I L-tartrate Form V.

FIG. 1C shows a thermogravimetric analysis (TGA) for Compound I L-tartrate Form V.

FIG. 1D shows a dynamic vapor sorption (DVS) curve for Compound I L-tartrate Form V.

FIG. 2A shows an XRPD pattern for Compound I L-tartrate Form VI.

FIG. 2B shows a DSC thermogram for Compound I L-tartrate Form VI.

FIG. 2C shows a TGA for Compound I L-tartrate Form VI.

FIG. 2D shows a DVS curve for Compound I L-tartrate Form VI.

### DETAILED DESCRIPTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The term “solvate” refers to a complex formed by combination of solvent molecules with molecules or ions of the solute. The solvent can be an organic compound, an inorganic compound, or a mixture of both. As used herein, the term “solvate” includes a “hydrate” (i.e., a complex formed by combination of water molecules with molecules or ions of the solute), hemi-hydrate, channel hydrate, etc. Some examples of solvents include, but are not limited to, acetonitrile, methanol, N,N-dimethylformamide, tetrahydrofuran, 2-methyltetrahydrofuran, dimethylsulfoxide, and water. In general, the solvated forms are equivalent to unsolvated forms and are encompassed within the scope of the present disclosure.

The term “desolvated” refers to a form that is a solvate as described herein, and from which solvent molecules have been partially or completely removed. Desolvation techniques to produce desolvated forms include, without limitation, exposure of a form (solvate) to a vacuum, subjecting the solvate to elevated temperature, exposing the solvate to a stream of gas, such as air or nitrogen, or any combination thereof. Thus, a desolvated or “unsolvated” form can be “anhydrous”, i.e., completely without solvent molecules, or partially solvated wherein solvent molecules are present in stoichiometric or non-stoichiometric amounts.

The term “amorphous” refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (glass transition).

“Substantially pure (form of a polymorph),” in some embodiments, means that in the referenced material, at least 99.9% of the material is the referenced polymorph. “Substantially pure (form of a polymorph),” in some embodiments, means that in the referenced material, at least 99.5% of the material is the referenced polymorph. “Substantially pure (form of a polymorph),” in some embodiments, means that in the referenced material, at least 99% of the material is the referenced polymorph. “Substantially pure form (of a polymorph),” in some embodiments, means that in the referenced material, at least 98% of the material is the referenced polymorph. “Substantially pure (form of a polymorph),” in some embodiments, means that in the referenced material, at least 97% of the material is the referenced polymorph. “Substantially pure (form of a polymorph),” in some embodiments, means that in the referenced material, at least 96% of the material is the referenced polymorph. “Substantially pure (form of a polymorph),” in some embodiments, means that in the referenced material, at least 95% of the material is the referenced polymorph.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables. The term “pharmaceutically acceptable salt” of a given compound refers to a salt that retains the biological effectiveness and properties of the given compound, and which are not biologically or otherwise undesirable. The term “pharmaceutically acceptable solvate” of a given compound likewise refers to a solvate of a given compound or salt thereof that retains the biological effectiveness and properties of the given compound or salt thereof, and which are not biologically or otherwise undesirable.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

The term “substantially the same as” or “substantially as shown” as used herein, refers to a powder X-ray diffraction pattern, DSC thermogram, TGA pattern, or DVS curve that is identical or non-identical to those depicted herein, but that falls within the limits of experimental error, when considered by one of ordinary skill in the art.

### Compound 1

Disclosed herein is 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), or a pharmaceutically acceptable salt or solvate thereof. Compound 1 refers to the compound with the following formula:

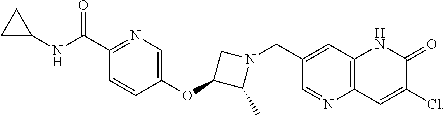 [SMILES: C[C@@H]1[C@@H](Oc2ccc(C(=O)NC3CC3)nc2)CN1Cc1cnc2cc(Cl)c(=O)[nH]c2c1]

Compound 1 refers to the compound with the following name: 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide.

While not intending to be bound by any particular theory, certain solid forms are characterized by physical properties, e.g., stability, solubility, and dissolution rate, appropriate for pharmaceutical and therapeutic dosage forms. Moreover, while not wishing to be bound by any particular theory, certain solid forms are characterized by physical properties (e.g., density, compressibility, hardness, morphology, cleavage, stickiness, solubility, water uptake, electrical properties, thermal behavior, solid-state reactivity, physical stability, and chemical stability) affecting particular processes (e.g., yield, filtration, washing, drying, milling, mixing, tableting, flowability, dissolution, formulation, and lyophilization) which make certain solid forms suitable for the manufacture of a solid dosage form. Such properties can be determined using particular analytical chemical techniques, including solid-state analytical techniques (e.g., X-ray diffraction, microscopy, spectroscopy, and thermal analysis), as described herein.

### Crystalline Forms

The identification and selection of a solid form of a pharmaceutical compound are complex, given that a change in solid form may affect a variety of physical and chemical properties, which may provide benefits or drawbacks in processing, formulation, stability, bioavailability, storage, and handling (e.g., shipping), among other important pharmaceutical characteristics. Useful pharmaceutical solids include crystalline solids and amorphous solids, depending on the product and its mode of administration. Amorphous solids are characterized by a lack of long-range structural order, whereas crystalline solids are characterized by structural periodicity. The desired class of pharmaceutical solid depends upon the specific application; amorphous solids are sometimes selected on the basis of, e.g., an enhanced dissolution profile, while crystalline solids may be desirable for properties such as, e.g., physical, or chemical stability.

Whether crystalline or amorphous, solid forms of a pharmaceutical compound include single-component and multiple-component solids. Single-component solids consist essentially of the pharmaceutical compound or active ingredient in the absence of other compounds. Variety among single-component crystalline materials may potentially arise from the phenomenon of polymorphism, wherein multiple three-dimensional arrangements exist for a particular pharmaceutical compound.

Notably, it is not possible to predict a priori if crystalline forms of a compound even exist, let alone how to successfully prepare them (see, e.g., Braga and Grepioni, 2005, “Making crystals from crystals: a green route to crystal engineering and polymorphism,” Chem. Commun.:3635-3645 (with respect to crystal engineering, if instructions are not very precise and/or if other external factors affect the process, the result can be unpredictable); Jones et al., 2006, “Pharmaceutical Cocrystals: An Emerging Approach to Physical Property Enhancement,” MRS Bulletin 31:875-879 (At present it is not generally possible to computationally predict the number of observable polymorphs of even the simplest molecules); Price, 2004, “The computational prediction of pharmaceutical crystal structures and polymorphism,” Advanced Drug Delivery Reviews 56:301-319 (“Price”); and Bernstein, 2004, “Crystal Structure Prediction and Polymorphism,” ACA Transactions 39:14-23 (a great deal still needs to be learned and done before one can state with any degree of confidence the ability to predict a crystal structure, much less polymorphic forms)).

The variety of possible solid forms creates potential diversity in physical and chemical properties for a given pharmaceutical compound. The discovery and selection of solid forms are of great importance in the development of an effective, stable, and marketable pharmaceutical product.

### Crystalline Forms of Compound 1

The polymorphs made according to the methods of the disclosure may be characterized by any methodology according to the art. For example, the polymorphs made according to the methods of the disclosure may be characterized by X-ray powder diffraction (XRPD), differential scanning calorimetry (DSC), thermogravimetric analysis (TGA), hot-stage microscopy, and/or spectroscopy (e.g., Raman, solid state nuclear magnetic resonance (ssNMR), and infrared (IR)). In some embodiments, crystallinity of a solid form is determined by X-Ray Powder Diffraction (XRPD).

XRPD: Polymorphs according to the disclosure may be characterized by XRPD. The relative intensities of XRPD peaks can vary, depending upon the particle size, the sample preparation technique, the sample mounting procedure and the particular instrument employed. Moreover, instrument variation and other factors can affect the 20 values. Therefore, the XRPD peak assignments can vary, for example by plus or minus 0.2 degrees or by plus or minus 0.1 degrees.

DSC: Polymorphs according to the disclosure can also be identified by its characteristic DSC thermograms. For DSC, it is known that the temperatures observed will depend upon the rate of temperature change as well as sample preparation technique and the particular instrument employed. Thus, the values reported herein relating to DSC thermograms can vary, for example by plus or minus 4° C.

TGA: The polymorphic forms of the disclosure may also give rise to thermal behavior different from that of the amorphous material or another polymorphic form. Thermal behavior may be measured in the laboratory by thermogravimetric analysis (TGA) which may be used to distinguish some polymorphic forms from others. In one aspect, the polymorph may be characterized by thermogravimetric analysis.

The polymorph forms of Compound 1 are useful in the production of medicinal preparations and can be obtained by means of a crystallization process to produce crystalline and semi-crystalline forms or a solidification process to obtain the amorphous form. In some embodiments, the crystallization is carried out by either generating the desired compound (for example, Compound 1) in a reaction mixture and isolating the desired polymorph from the reaction mixture, or by dissolving raw compound in a solvent, optionally with heat, followed by crystallizing/solidifying the product by cooling (including active cooling) and/or by the addition of an antisolvent for a period of time. In some embodiments, the crystallization comprises addition of a seed form of a desired polymorph. The crystallization or solidification may be followed by drying carried out under controlled conditions until the desired water content is reached in the end polymorphic form.

### Forms of Compound I

### Compound I L-Tartrate Form V

In some embodiments, provided herein is crystalline 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide L-tartrate Form V (Compound I L-tartrate Form V) characterized by an X-ray powder diffractogram comprising peaks (±0.2°) at 5.2, 10.3, and 15.5° 2θ as determined on a diffractometer using Cu-Kα radiation.

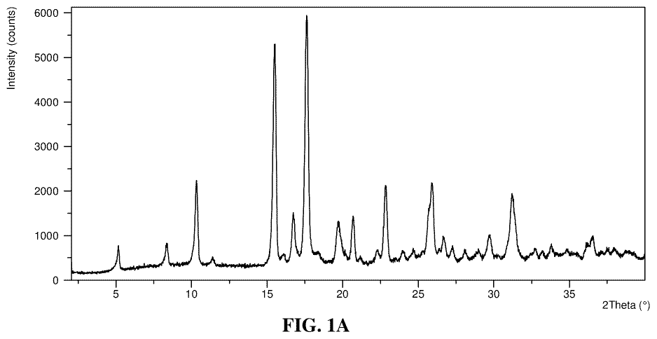

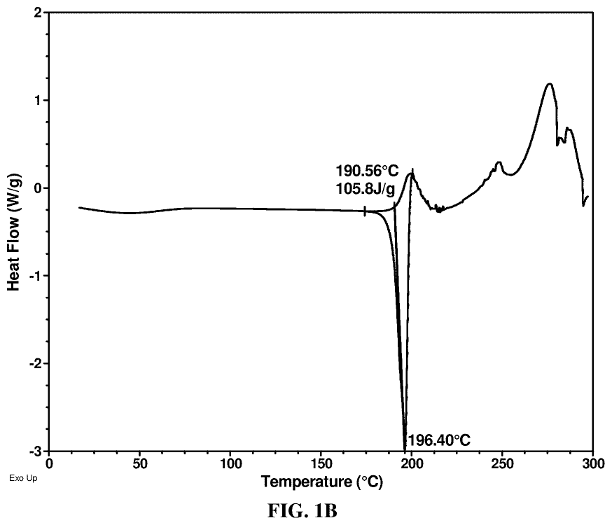

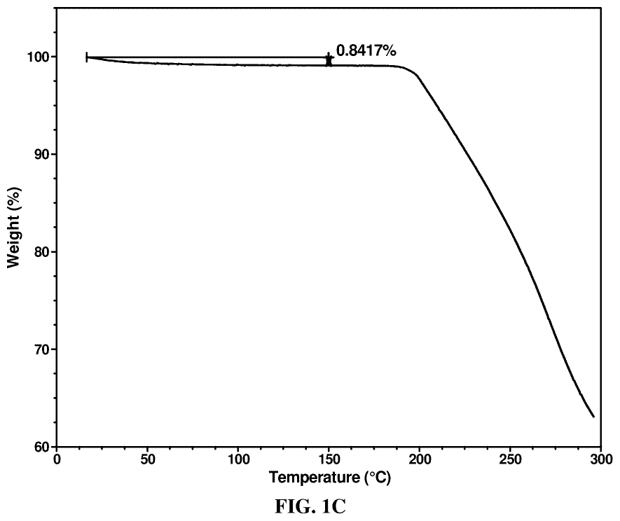

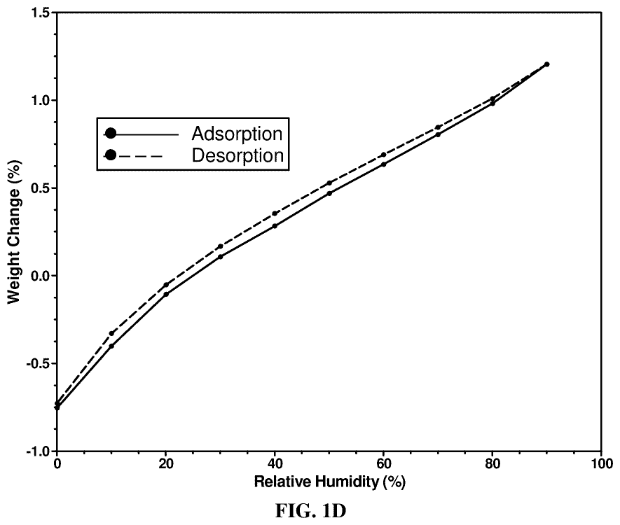

In some embodiments, Compound I L-tartrate Form V is further characterized by:
i) one or more peaks at (±0.2°) at 8.4, 16.7, and 17.7° 2θ;
ii) a diffractogram substantially as shown in FIG. 1A;
iii) a differential scanning calorimetry (DSC) curve comprising an endotherm at about 191° C. (onset temperature);
iv) a differential scanning calorimetry (DSC) curve substantially as shown in FIG. 1B;
v) thermogravimetric analysis (TGA) showing a weight loss of about 0.8 wt % from 25 to 150° C.;
vi) thermogravimetric analysis (TGA) comprising a thermogram substantially as shown in FIG. 1C; or
vii) a dynamic vapor sorption (DVS) curve showing about 2% water uptake from 0 to 90% relative humidity (RH) at 25° C.; or
viii) a DVS curve substantially as shown in FIG. 1D.

In some embodiments, Compound I L-tartrate Form V is further characterized by an X-ray powder diffractogram comprising one or more peaks at (±0.2°) at 8.4, 16.7, and 17.7° 2θ as determined on a diffractometer using Cu-Kα radiation. In some embodiments, Compound I L-tartrate Form V is further characterized by an X-ray powder diffractogram comprising one or more peaks at (±0.2°) at 20.7, 22.9, and 25.7° 2θ as determined on a diffractometer using Cu-Kα radiation. In some embodiments, Compound I L-tartrate Form V is further characterized by a diffractogram substantially as shown in FIG. 1A.

In some embodiments, Compound I L-tartrate Form V is further characterized by a DSC curve comprising an endotherm at about 191° C. (onset temperature). In some embodiments, Compound I L-tartrate Form V is further characterized by a DSC curve comprising an endotherm at about 196° C. (peak).

In some embodiments, Compound I L-tartrate Form V is further characterized by a DSC curve substantially as shown in FIG. 1B.

In some embodiments, Compound I L-tartrate Form V is further characterized by TGA showing a weight loss of about 0.8 wt % from 25 to 150° C. In some embodiments, Compound I L-tartrate Form V is further characterized by TGA comprising a thermogram substantially as shown in FIG. 1C.

In some embodiments, Compound I L-tartrate Form V is further characterized by a DVS curve showing about 2% water uptake from 0 to 90% relative humidity (RH) at 25° C. In some embodiments, Compound I L-tartrate Form V is further characterized by a DVS curve substantially as shown in FIG. 1D.

### Compound I L-tartrate Form VI

In some embodiments, provided herein is crystalline 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide L-tartrate Form VI (Compound I L-tartrate Form VI) characterized by an X-ray powder diffractogram comprising peaks (±0.2°) at 8.6, 9.6, and 17.1° 2θ as determined on a diffractometer using Cu-Kα radiation.

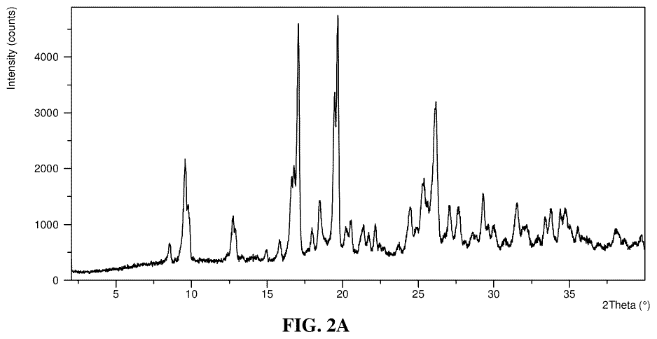

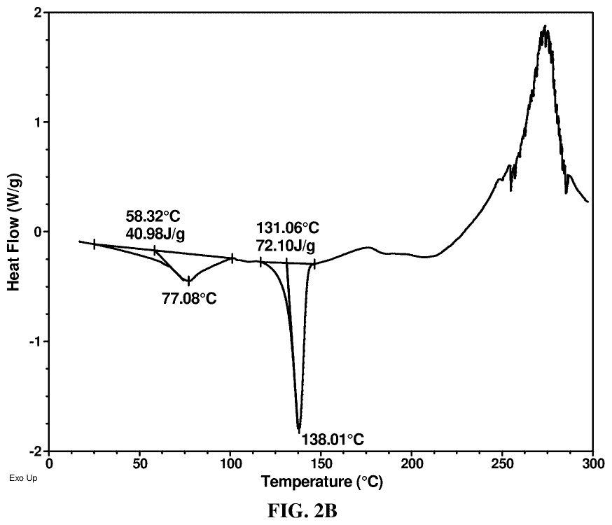

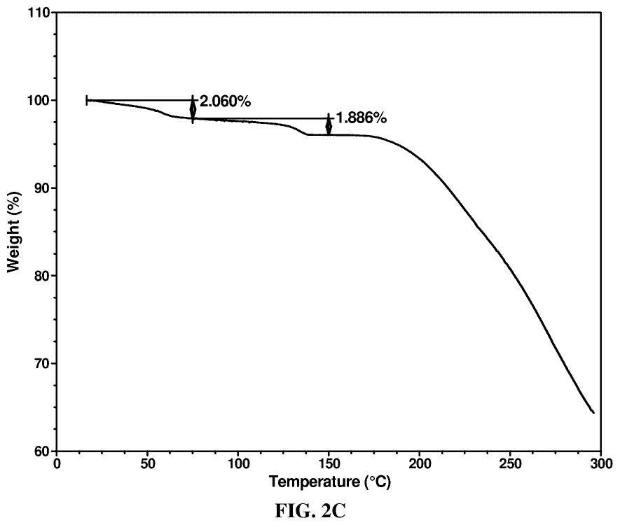

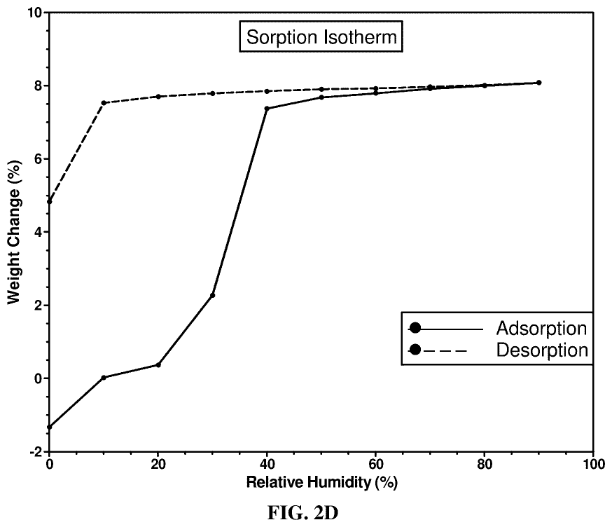

In some embodiments, Compound I L-tartrate Form VI is further characterized by:
i) one or more peaks at (±0.2°) at 16.6, 18.5, and 19.7° 2θ;
ii) a diffractogram substantially as shown in FIG. 2A;
iii) a differential scanning calorimetry (DSC) curve comprising an endotherm at about 58° C. (onset temperature) and an endotherm at about 131° C. (onset temperature);
iv) a differential scanning calorimetry (DSC) curve substantially as shown in FIG. 2B;
v) thermogravimetric analysis (TGA) showing a weight loss of about 2% from 25 to 75° C. and about 1.9% from 75 to 150° C.;
vi) thermogravimetric analysis (TGA) comprising a thermogram substantially as shown in FIG. 2C; or
vii) a dynamic vapor sorption (DVS) curve showing about 9% water uptake from 0 to 90% relative humidity (RH) at 25° C.; or
viii) a DVS curve substantially as shown in FIG. 2D.

In some embodiments, Compound I L-tartrate Form VI is characterized by an X-ray powder diffractogram comprising one or more peaks at (±0.2°) at 16.6, 18.5, and 19.7° 2θ as determined on a diffractometer using Cu-Kα radiation. In some embodiments, Compound I L-tartrate Form VI is characterized by an X-ray powder diffractogram comprising one or more peaks at (±0.2°) at 25.3, 26.2, and 29.3° 2θ as determined on a diffractometer using Cu-Kα radiation. In some embodiments, Compound I L-tartrate Form VI is further characterized by a diffractogram substantially as shown in FIG. 2A.

In some embodiments, Compound I L-tartrate Form VI is further characterized by a DSC curve comprising an endotherm at about 58° C. (onset temperature) and an endotherm at about 131° C. (onset temperature). In some embodiments, Compound I L-tartrate Form VI is further characterized by a DSC curve comprising an endotherm at about 77° C. (peak) and an endotherm at about 138° C. (peak). In some embodiments, Compound I L-tartrate Form VI is further characterized by a DSC curve substantially as shown in FIG. 2B.

In some embodiments, Compound I L-tartrate Form VI is further characterized by TGA showing a weight loss of about 2% from 25 to 75° C. and about 1.9% from 75 to 150° C. In some embodiments, Compound I L-tartrate Form VI is further characterized by TGA comprising a thermogram substantially as shown in FIG. 2C.

In some embodiments, Compound I L-tartrate Form VI is further characterized by a DVS curve showing about 2% water uptake from 0 to 90% relative humidity (RH) at 25° C. In some embodiments, Compound I L-tartrate Form VI is further characterized by a DVS curve substantially as shown in FIG. 2D.

In another embodiment, crystalline Compound 1 as described herein is substantially pure. In some embodiments, the substantially pure crystalline Compound 1 is substantially free of other solid forms, e.g., amorphous solid. In some embodiments, the purity of the substantially pure crystalline Compound 1 is no less than about 95%, no less than about 96%, no less than about 97%, no less than about 98%, no less than about 98.5%, no less than about 99%, no less than about 99.5%, or no less than about 99.8%. In some embodiments, the purity of the substantially pure crystalline Compound 1 is about 95%, about 96%, about 97%, about 98%, about 98.5%, about 99%, about 99.5%, or about 99.8%.

### Method of Treatment

Disclosed herein are methods of treatment of a disease in which inhibition of PARP is beneficial, the method comprising administering a compound disclosed herein. Also disclosed herein are methods of treatment of a disease in which inhibition of PARP1 is beneficial, the method comprising administering a compound disclosed herein. In some embodiments, the disease is cancer. In some embodiments, the cancer is breast cancer, ovarian cancer, pancreatic cancer, prostate cancer, a hematological cancer, a gastrointestinal cancer such as gastric cancer and colorectal cancer, or lung cancer. In some embodiments, the cancer is breast cancer, ovarian cancer, pancreatic cancer, or prostate cancer. In some embodiment, the cancer is leukemia, colon cancer, glioblastoma, lymphoma, melanoma, or cervical cancer.

In some embodiments, the cancer comprises a BRCA1 and/or a BRCA2 mutation.

In some embodiments, the cancer comprising a BRCA1 and/or a BRCA2 mutation is bladder cancer, brain & CNS cancers, breast cancer, cervical cancer, colorectal cancer, esophagus cancer, Hodgkin lymphoma, non-Hodgkin lymphoma, kidney cancer, leukemia, lung cancer, melanoma, myeloma, oral cavity cancer, ovarian cancer, pancreatic cancer, prostate cancer, skin cancer, stomach cancer, thyroid cancer, or uterus cancer.

In some embodiments, the cancer is a cancer deficient in Flomologous Recombination (FIR) dependent DNA DSB repair activity. The FIR dependent DNA DSB repair pathway repairs double-strand breaks (DSBs) in DNA via homologous mechanisms to reform a continuous DNA helix. The components of the FIR dependent DNA DSB repair pathway include, but are not limited to, ATM (NM_000051), RAD51 (NM_002875), RAD51 L1 (NM_002877), RAD51 C (NM_002876), RAD51 L3 (NM_002878), DMC1 (NM_007068), XRCC2 (NM_005431), XRCC3 (NM_005432), RAD52 (NM_002879), RAD54L (NM_003579), RAD54B (NM_012415), BRCA1 (NM_007295), BRCA2 (NM_000059), RAD50 (NM_005732), MRE1 1 A (NM_005590) and NBS1 (NM_002485). Other proteins involved in the FIR dependent DNA DSB repair pathway include regulatory factors such as EMSY. In some embodiments, the cancer which is deficient in FIR dependent DNA DSB repair comprises one or more cancer cells which have a reduced or abrogated ability to repair DNA DSBs through that pathway, relative to normal cells i.e. the activity of the FIR dependent DNA DSB repair pathway may be reduced or abolished in the one or more cancer cells.

In some embodiments, the activity of one or more components of the FIR dependent DNA DSB repair pathway is abolished in the one or more cancer cells of an individual having a cancer which is deficient in FIR dependent DNA DSB repair.

In some embodiments, the cancer cells have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2, i.e. expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor. BRCA1 and BRCA2 are known tumor suppressors whose wild-type alleles are frequently lost in tumors of heterozygous carriers. Amplification of the EMSY gene, which encodes a BRCA2 binding factor, is also known to be associated with breast and ovarian cancer. Carriers of mutations in BRCA1 and/or BRCA2 are also at elevated risk of certain cancers, including breast cancer, ovarian cancer, pancreatic cancer, prostate cancer, a hematological cancer, gastrointestinal cancer, and lung cancer.

To minimize the risks of off-target effects, it is desirable for drug molecules to possess selectivity for a specific target.

Avoiding inhibition of PARP family isoforms beyond PARP1 may be important in minimizing toxicities that may arise from inhibition of non-PARP1 isoforms. The pharmacology of inhibiting PARP isoforms beyond PARP1 may drive toxicities that reduce the therapeutic index for agents that possess lower selectivity's for PARP1 against PARP isoforms. PARP3, like PARP1, plays a role in DNA damage but has also been found to be a key player in the integrity of the mitotic spindle and in telomerase integrity (Boehler, C. Gauthier, L R., Mortusewicz O, et al. Poly(ADP-ribose) polymerase 3 (PARP3), a newcomer in cellular response to DNA damage and mitotic progression. PNAS, Jan. 26, 2011, 108 (7) 2783-2788). PARP5A also known as Tankyrase 1, plays key roles in Wnt signaling and telomere length (Kulak, O., Chen. H., Holohan B. et al. Disruption of Wnt/β-Catenin Signaling and Telomeric Shortening Are Inextricable Consequences of Tankyrase Inhibition in Human Cells. Mol Cell Biol. 2015 Jul; 35(14), 2425-2435). PARP6 is an essential microtubule-regulatory gene in mice, germline mutations in PARP6 that abrogate the catalytic activity has negative effects on neuronal function in humans (Vermehren-Schmaedick, A., Huang J. Y,, Levinson, M. et al. Characterization of PARP6 Function in Knockout Mice and Patients with Developmental Delay. Cells, 2021 Jun; 10(6), 1289). PARP7 catalytic inhibition causes hyper stimulatory effects on type one interferon producing an autoimmune phenotype (Gozgit, J. M., Vasbinder, M. M., Abo, R. P. et al. PARP7 negatively regulates the type I interferon response in cancer cells and its inhibition triggers antitumor immunity, Volume 39, Issue 9, 13 Sep. 2021, Pages 1214-1226). While the exact function of PARP8 has not been established, its knockout has been shown to induce mitotic and nuclear morphology defects and a decrease in cellular viability (Vyas, S., Chesarone-Cataldo, M., Todorova, T., et al. A Systematic Analysis of the PARP Protein Family Identifies New Functions Critical for Cell Physiology. Nat. Commun. 2013, 4 (1), 2240) PARP10 has been described as a MYC interacting protein with tunor suppressor activities (Yu, M. Schreek, S., Cerni, C. et al. PARP-10, a novel Myc-interacting protein with poly(ADP-ribose) polymerase activity, inhibits transformation. Oncogene, 2005 volume 24, pages1982-1993).

### Dosing

In certain embodiments, the compositions containing Compound 1, or a pharmaceutically acceptable salt or solvate thereof, are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In certain embodiments wherein the patient's condition does not improve, upon the doctor's discretion, the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage, or the frequency of administration, or both, is reduced, as a function of the symptoms, to a level at which the improved disease, disorder or condition is retained. In certain embodiments, however, the patient requires intermittent or daily treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage, or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₁₀ and the ED₉₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, Compound 1, or a pharmaceutically acceptable salt or solvate thereof, is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

### Compositions/Pharmaceutical Compositions/Formulations

In some embodiments, provided is a composition comprising a salt or solid form of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), as described herein.

In one embodiment, provided is a composition comprising a salt or solid form of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound 1 present in a composition is in the designated salt, solid form, crystalline form, or crystalline salt form.

In one embodiment, provided is a composition comprising a salt or solid form of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), or solvate thereof, wherein at least 95% of Compound 1 present in a composition is in the designated salt, solid form, crystalline form, or crystalline salt form.

In one embodiment, provided is a composition comprising a salt or solid form of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), or solvate thereof, wherein at least 90% of Compound 1 present in a composition is in the designated salt, solid form, crystalline form, or crystalline salt form.

In one embodiment, provided is a composition comprising a salt or solid form of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), or solvate thereof, wherein at least 85% of Compound 1 present in a composition is in the designated salt, solid form, crystalline form, or crystalline salt form.

In one embodiment, provided is a pharmaceutical composition comprising a salt or solid form of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), or solvate thereof, wherein at least 95% of Compound 1 present in a pharmaceutical composition is in the designated salt, solid form, crystalline form, or crystalline salt form.

In one embodiment, provided is a pharmaceutical composition comprising a salt or solid form of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), or solvate thereof, wherein at least 90% of Compound 1 present in a pharmaceutical composition is in the designated salt, solid form, crystalline form, or crystalline salt form.

In one embodiment, provided is a pharmaceutical composition comprising a salt or solid form of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), or solvate thereof, wherein at least 85% of Compound 1 present in a pharmaceutical composition is in the designated salt, solid form, crystalline form, or crystalline salt form.

In some embodiments, Compound 1, or a pharmaceutically acceptable salt or solvate thereof, is administered to a subject in need thereof, either alone or in combination with pharmaceutically acceptable carriers, excipients, or diluents, in a pharmaceutical composition, according to standard pharmaceutical practice. In one embodiment, Compound 1, or a pharmaceutically acceptable salt or solvate thereof, may be administered to animals. Compound 1, or a pharmaceutically acceptable salt or solvate thereof, can be administered orally or parenterally, including the intravenous, intramuscular, intraperitoneal, subcutaneous, rectal, and topical routes of administration.

In another aspect, provided herein are pharmaceutical compositions comprising Compound 1, or a pharmaceutically acceptable salt or solvate thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

### Combination

Disclosed herein are methods of treating cancer using Compound 1, or a pharmaceutically acceptable salt or solvate thereof, in combination with an additional therapeutic agent.

In some embodiments, the additional therapeutic agent is an anticancer agent.

In some embodiments, the additional therapeutic agent is administered at the same time as Compound 1, or a pharmaceutically acceptable salt or solvate thereof. In some embodiments, the additional therapeutic agent and Compound 1, or a pharmaceutically acceptable salt or solvate thereof, are administered sequentially. In some embodiments, the additional therapeutic agent is administered less frequently than Compound 1, or a pharmaceutically acceptable salt or solvate thereof. In some embodiments, the additional therapeutic agent is administered more frequently than Compound 1, or a pharmaceutically acceptable salt or solvate thereof. In some embodiments, the additional therapeutic agent is administered prior to the administration of Compound 1, or a pharmaceutically acceptable salt or solvate thereof. In some embodiments, the additional therapeutic agent is administered after the administration of Compound 1, or a pharmaceutically acceptable salt or solvate thereof.

### EXAMPLES

### Example 1: Solid Forms of Compound I

### Analysis Methods

### Equipment

XRPD patterns were collected with a PANalytical Empyrean diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640e) was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning line detector, PIXcel1D-Medipix3 PASS (programmable anti-scatter slit), located 240 mm from the sample and Data Collector software v. 7.2b.

Differential Scanning Calorimetry (DSC) was run on Model Q2000 (TA Instruments, New Castle, DE). About 1-5 mg of material was loaded into a Tzero standard aluminum pan with a manually punctured pinhole on the lid. By default, the sample and reference pans were heated from 20 to 300° C. under nitrogen purge of 50 mL/min. DSC was run at a heating rate of 10° C./min. Data analysis was completed using Universal Analysis 2000 Version 4.5A (TA Instruments, New Castle, DE).

Thermogravimetric Analysis (TGA) was used to evaluate sample weight loss as a function of temperature on either Model Q5000 or Q500 (TA Instruments, New Castle, DE). About 1 to 5 mg of material was loaded onto a sample pan and the sample was heated from ambient temperature to 300° C. or above at a rate of 10° C./min. The sample pan was under nitrogen purge at 40 mL/min. Data analysis was completed using Universal Analysis 2000 Version 4.5A (TA Instruments, New Castle, DE).

Dynamic Vapor Sorption (DVS) was used to study hygroscopicity on Model Q5000 SA (TA Instruments, New Castle, DE). A sample (1-10 mg) was placed in an aluminum pan and loaded on the sample side of the twin pan balance. The water sorption and desorption were studied as a function of relative humidity (RH) at 25° C. in 10% RH increments from 0% RH to 90% RH and then back to 0%. Each relative humidity increment had an equilibration time of 120 minutes unless weight change was less than 0.002% in 20 minutes. Data analysis was performed using Universal Analysis 2000 Version 4.7A (TA Instruments, New Castle, DE).

Proton Nuclear Magnetic Resonance (¹H NMR) spectra were collected on a Bruker Avance III-HD 400 with SampleXpress. The default proton parameters are: spectral width: 16.19 to −3.84 ppm (8012.8 Hz); relaxation delay: 1 sec; pulse: 90 degrees; acquisition time: 4.0894 sec; number of scans or repetitions: 16; temperature: 25° C. Off-line analysis was conducted using MNova software.

### Compound I L-Tartrate Form V

About 3.0 g of free base Compound I Form I, which can be made according to methods described in WO 2024/206172, and 2.05 g of L-tartaric acid (about 2 equivalents (eq.)) were stirred in 75 mL of methanol (MeOH) at 50° C. for about 20 h to become a thick slurry. It was diluted with 25 mL of MeOH. XRPD showed some free base remaining. After stirring for 1 more day, 0.205 g of L-tartaric acid (0.2 eq.) was charged. After stirring for 1 h at 50° C., it was cooled to 20° C. After 1 hour, another portion of 0.205 g of L-tartaric acid was charged. Total amount of tartaric acid used in this preparation was 2.4 eq. (relative to free base). The sample was stirred for 2 h and about three-fourths of the slurry was filtered, washed with 10 mL of MeOH, and dried at 50° C. under vacuum. After drying for 4 day, XRPD analysis showed it has a unique pattern, designated as Compound I L-tartrate Form V. Proton NMR of the solid showed it contained 1 eq. L-tartaric acid and 0.01 eq. of MeOH.

A representative XRPD pattern of Compound I L-tartrate Form V is shown in FIG. 1A. The DSC thermogram indicated an endotherm with onset at about 191° C. (FIG. 1B). The TGA thermogram showed a weight loss of about 0.8% from 25 to 150° C. (FIG. 1C). DVS analysis showed that Compound I L-tartrate Form V was slightly hygroscopic, picking up about 2% water from 0 to 90% RH at 25° C. (FIG. 1D).

### Compound I L-Tartrate Form VI

About 0.2 g of Compound I L-tartrate Form I, which can be made according to methods described in WO 2024/206172, was stirred in a mixture of 1.088 mL of water and 0.812 mL of acetone at 22° C. for about 1 day. The sample was heated at 50° C. for 8 h, and then cooled to 22° C. After stirring for about 2 days, XRPD analysis showed the sample converted to Compound I L-tartrate Form II (as characterized in WO 2024/206172). After holding for 3 more days, the sample was filtered and dried in a vacuum oven at 100° C. for 4 h. XRPD showed it changed to Compound I L-tartrate Form VI.

A representative XRPD pattern of Compound I L-tartrate Form VI is shown in FIG. 2A. The DSC thermogram indicated two endotherms with onsets at about 58 and 131° C., respectively (FIG. 2B). The TGA thermogram showed two weight loss events of about 2% from 25 to 75° C., and 1.9% from 75 to 150° C., respectively (FIG. 2C). DVS analysis showed that Compound I L-tartrate Form VI was hygroscopic, picking up about 9% water from 0 to 90% RH at 25° C. (FIG. 2D). After DVS analysis, XRPD showed that it converted back to Compound I L-tartrate Form II.

1. A crystalline form of a L-tartrate salt of Compound I: 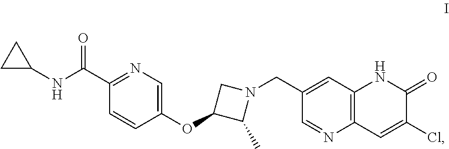 [SMILES: C[C@@H]1[C@@H](Oc2ccc(C(=O)NC3CC3)nc2)CN1Cc1cnc2cc(Cl)c(=O)[nH]c2c1]
(Compound I L-tartrate Form V), wherein Compound I L-tartrate Form V is characterized by an X-ray powder diffractogram comprising peaks (+0.2°) at 5.2, 10.3, and 15.5° 2θ as determined on a diffractometer using Cu-Kα radiation.

2. The crystalline form of claim 1, further characterized by:
i) one or more peaks at (±0.2°) at 8.4, 16.7, and 17.7° 2θ;
ii) a diffractogram substantially as shown in FIG. 1A;
iii) a differential scanning calorimetry (DSC) curve comprising an endotherm at about 191° C. (onset temperature);
iv) a differential scanning calorimetry (DSC) curve substantially as shown in FIG. 1B;
v) thermogravimetric analysis (TGA) showing a weight loss of about 0.8 wt % from 25 to 150° C.;
vi) thermogravimetric analysis (TGA) comprising a thermogram substantially as shown in FIG. 1C; or
vii) a dynamic vapor sorption (DVS) curve showing about 2% water uptake from 0 to 90% relative humidity (RH) at 25° C.; or
viii) a DVS curve substantially as shown in FIG. 1D.

3. A crystalline form of a L-tartrate salt of Compound I: 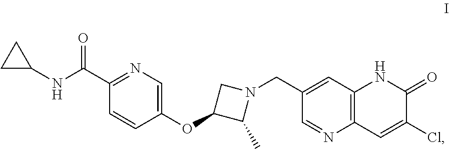 [SMILES: C[C@@H]1[C@@H](Oc2ccc(C(=O)NC3CC3)nc2)CN1Cc1cnc2cc(Cl)c(=O)[nH]c2c1]
(Compound I L-tartrate Form VI), wherein Compound I L-tartrate Form VI is characterized by an X-ray powder diffractogram comprising peaks (±0.2°) at 8.6, 9.6, and 17.1° 2θ as determined on a diffractometer using Cu-Kα radiation.

4. The crystalline form of claim 3, further characterized by:
i) one or more peaks at (±0.2°) at 16.6, 18.5, and 19.7° 2θ;
ii) a diffractogram substantially as shown in FIG. 2A;
iii) a differential scanning calorimetry (DSC) curve comprising an endotherm at about 58° C. (onset temperature) and an endotherm at about 131° C. (onset temperature);
iv) a differential scanning calorimetry (DSC) curve substantially as shown in FIG. 2B;
v) thermogravimetric analysis (TGA) showing a weight loss of about 2% from 25 to 75° C. and about 1.9% from 75 to 150° C.;
vi) thermogravimetric analysis (TGA) comprising a thermogram substantially as shown in FIG. 2C; or
vii) a dynamic vapor sorption (DVS) curve showing about 9% water uptake from 0 to 90% relative humidity (RH) at 25° C.; or
viii) a DVS curve substantially as shown in FIG. 2D.

5. A pharmaceutical composition comprising a crystalline form of claim 1, and a pharmaceutically acceptable excipient.

6. A method of treating cancer in a subject in need thereof, the method comprising administering a crystalline form of claim 1.

7. The method of claim 6, wherein the cancer is breast cancer, ovarian cancer, pancreatic cancer, prostate cancer, a hematological cancer, gastrointestinal cancer, or lung cancer.

8. A method of treating a cancer comprising a BRCA1 and/or a BRCA2 mutation in a subject in need thereof, the method comprising administering a crystalline form of claim 1.

9. The method of claim 8, wherein the cancer is bladder cancer, brain & CNS cancers, breast cancer, cervical cancer, colorectal cancer, esophagus cancer, Hodgkin lymphoma, non-Hodgkin lymphoma, kidney cancer, leukemia, lung cancer, melanoma, myeloma, oral cavity cancer, ovarian cancer, pancreatic cancer, prostate cancer, skin cancer, stomach cancer, thyroid cancer, or uterus cancer.

10. A pharmaceutical composition comprising a crystalline form of claim 3, and a pharmaceutically acceptable excipient.

11. A method of treating cancer in a subject in need thereof, the method comprising administering a crystalline form of claim 3.

12. The method of claim 11, wherein the cancer is breast cancer, ovarian cancer, pancreatic cancer, prostate cancer, a hematological cancer, gastrointestinal cancer, or lung cancer.

13. A method of treating a cancer comprising a BRCA1 and/or a BRCA2 mutation in a subject in need thereof, the method comprising administering a crystalline form of claim 3.

14. The method of claim 13, wherein the cancer is bladder cancer, brain & CNS cancers, breast cancer, cervical cancer, colorectal cancer, esophagus cancer, Hodgkin lymphoma, non-Hodgkin lymphoma, kidney cancer, leukemia, lung cancer, melanoma, myeloma, oral cavity cancer, ovarian cancer, pancreatic cancer, prostate cancer, skin cancer, stomach cancer, thyroid cancer, or uterus cancer.
